(19) United States
(12) Patent Application Publication
(10) Pub. No.: US 2026/0227352 A1
(43) Pub. Date: Aug. 6, 2026

(54) IN-LINE SOLIDS DEPOSITION SENSOR

(71) Applicant: The University of Western Australia, Crawley, Western Australia (AU)

(72) Inventors: Paul Louis STANWIX, Crawley, Western Australia (AU); Matthew Gaven HOPKINS, Crawley, Western Australia (AU); Eric Freemantle MAY, Crawley, Western Australia (AU)

(73) Assignee: The University of Western Australia, Crawley, Western Australia (AU)

(21) Appl. No.: 19/151,596

(22) PCT Filed: Jan. 25, 2024
(86) PCT No.: PCT/AU2024/050043

§ 371 Date: Jul. 28, 2025

(30) Foreign Application Priority Data
Jan. 27, 2023 (AU) 2023900190

(51) Int. Cl. G01N 25/12; G01B 15/02; G01N 22/00

(52) U.S. Cl. CPC G01N 25/12; G01B 15/02; G01N 22/00

(57) ABSTRACT

An in-line sensor for detecting solids deposition in a conduit is described. The sensor includes a body positionable to define at least a segment of a flow path for a fluid in or contiguous with the conduit; a cavity in the body which defines an electromagnetic resonant cavity that operates at frequencies up to and including microwave frequencies, with resonant properties sensitive to the presence of a solid phase deposited on a conduit surface. The sensor also includes one or more probes for exciting and monitoring an electromagnetic resonance of the cavity; and a signal processor operatively connected to said one or more probes to directly detect solids deposition on the conduit surface in response to detected changes in the resonant properties of the cavity. The sensor may be used to detect the composition and or thickness of the solid deposit in the conduit.

### CROSS-REFERENCE TO RELATED APPLICATIONS

The present disclosure claims priority to the Australia Patent Application No. 2023900190 filed with the Australia Patent Office on Jan. 27, 2023 and entitled “IN-LINE SOLIDS DEPOSITION SENSOR”, the contents of which are incorporated herein by reference in entirety.

### TECHNICAL FIELD

The disclosure relates to an in-line solids sensor for detecting the presence of solids, such as gas hydrates or waxes, in pipelines.

### BACKGROUND

The discussion of the background to the disclosure is intended to facilitate an understanding of the disclosure. However, it should be appreciated that the discussion is not an acknowledgement or admission that any of the material referred to was published, known or part of the common general knowledge as at the priority date of the application.

Natural gas is playing an increasingly important role in world energy use and has been identified as a key transition fuel towards lower emission energy production. Beyond its direct use, it presents as a promising feedstock for hydrogen synthesis through methane reforming with carbon capture and sequestration (CCS).

Consequently, there has been continued growth in the sector which has facilitated development of offshore and onshore natural gas infrastructure. An ever-present risk associated with these developments is the primary fouling mechanism of gas (clathrate) hydrate formation and deposition. Gas hydrates are ice-like compounds where water molecules enclathrate a guest gas species. Facilitated by high gas pressures, these compounds can form at moderate temperatures (0 to 20° C.) thus exposing gas assets to flow assurance risks if not actively managed. Points of exposure include but are not limited to (i) subsea pipelines in heat exchange with cold ocean environments, (ii) Joule-Thomson cooling sites subsea or onshore, and (iii) active cooling of water-gas streams with refrigeration systems. An example subsea pipeline profile is depicted in FIG. 1 whereby approximately 70% of the pipe exists in a region of hydrate stability for an uninhibited system.

Typical hydrate management techniques include active prevention via dosing of thermodynamic hydrate inhibitors (e.g., monoetheylene glycol, methanol) to shift the equilibrium formation temperature below operating conditions. However, with the increasing development of long-distance subsea tiebacks, there is a decrease in economic viability for complete hydrate avoidance resulting in a paradigm shift that relies on accurate predictions of hydrate growth and transportability. The conceptual plugging mechanism for gas dominant systems is represented in FIG. 2, beginning with phase dispersion and hydrate film growth, followed by shear breakup and subsequent plug formation. Ideally, if operators could detect initial hydrate film growth, reactive measures could be implemented to remove the deposit before sufficient hydrate formed to the detriment of product throughput or safety.

Industrially, it is technically challenging to investigate the internal conditions of a pipeline without the need for physical intrusion or shutdown and inspection. There are several methods for detecting hydrate deposition. Acoustic techniques rely on propagating sound waves through the material and measuring signal attenuation and reflections, being indicative of deposit properties. While this technique is non-intrusive and capable of measuring composition and thickness of the deposit, it is limited for flow conditions within the pipeline. Distributed pressure techniques measure light scatter influenced by the strain on fiber optic cables. This technique is intrusive and is only capable of measuring the thickness of the deposit. Gamma ray techniques utilize the absorption of radiation to image the contents of the pipe. This is a mature and accurate technology that can measure the composition and thickness of the deposit. However, it is expensive and does not provide information in real time. Each of the above techniques are inherently implicit in their analysis and limited by a lack of resolution until significant changes in composition or large occlusions occur.

The present disclosure provides an in-line solids sensor that seeks to overcome at least some of the disadvantages of existing techniques for sensing and measuring the formation of solids such as gas hydrates or waxes on interior surfaces of a pipeline.

### SUMMARY

The disclosure provides an in-line solids sensor for detecting solids deposition, such as gas hydrates or waxes, in pipelines.

One aspect of the disclosure provides an in-line sensor for detecting solids deposition in a conduit, the sensor comprising:
a body positionable to define at least a segment of a flow path for a fluid in or contiguous with the conduit;
a cavity in the body defining an electromagnetic resonant cavity, operating at frequencies up to and including microwave frequencies, with resonant properties sensitive to the presence of a solid phase deposited on a conduit surface;
one or more probes for exciting and monitoring an electromagnetic resonance of the cavity; and
a signal processor operatively connected to said one or more probes to directly detect solids deposition on the conduit surface in response to detected changes in the resonant properties of the cavity.

The phrase “directly detect solids deposition” as used herein refers to the ability to sense or measure a change in a physical property caused by the presence of a solids deposit. Direct detection of a solids deposit may be distinguished from indirect detection whereby an apparatus, instrument or sensor is capable of measuring or sensing a physical property, such as temperature or pressure, at which solids are predicted or anticipated to form a deposit.

In one embodiment, the cavity comprises an annular chamber having an annular groove radially extending therefrom to the conduit surface. In one form, the annular chamber may be toroidal having an internal radius ro. In another form, the annular chamber may have a square or rectangular cross-section, optionally with curved corners. In another form, the annular chamber may be elliptical.

In one form, a width t of the annular groove may be shorter than the length w of the groove from the annular chamber to the conduit surface.

In one embodiment, said body comprises a pair of annular flanges each contiguous with a respective conduit section, wherein opposing faces of said flanges cooperate to define the cavity when the flanges are assembled together. The body may comprise suitable through-holes in the annular flanges and matching bolts are provided for clamping the flanges together.

In one form of this particular embodiment, the opposing face of one of said flanges may be flat, an outer annular recess and an inner annular recess being provided in the opposing face of the other of said flanges.

In another form of this particular embodiment, the opposing faces of said flanges are provided with an outer annular circumferential recess and an inner annular flat-sided recess.

In another embodiment, said body comprises a toroidal body, wherein opposing sides of the toroidal body are contiguous with a respective conduit section.

In an alternative embodiment, said body comprises a flange mountable plate having suitable through-holes in said plate and matching bolts to clamp the flange mountable plate to an end surface of the conduit. In this particular embodiment, said plate may be provided with a plurality of concentric toroidal cavities. For example, a first toroidal cavity may comprise a first annular chamber having a first annular groove radially extending therefrom to an exposed plate surface and a second toroidal cavity may comprise second concentric annular chamber having a second annular groove radially extending therefrom to the exposed plate surface. The second concentric annular chamber may have the same or different dimensions to the first annular chamber. The second annular groove may have the same or different depth to the first annular groove. The second annular groove may have the same or different width to the first annular groove.

In one embodiment, the cavity in the body may be filled with a non-conductive material capable of forming a gas tight seal. Suitable examples include, but are not limited to PTFE, PEEK, neoprene, nitrile, viton, fluorosilicone, glass-filled PTFE.

In one embodiment, said sensor is capable of detecting a solids deposit having a height in a range of 10 μm to 8 mm and a width in a range of 10 μm to 8 mm.

In one embodiment, said signal processor may be arranged to directly detect solids deposition on the conduit surface in response to detected changes to dielectric permittivity (As) with time.

In another aspect, the disclosure provides a method of detecting solids deposition in a conduit, the method comprising the steps of:
positioning a body to define at least a segment of a flow path for a fluid in or contiguous with the conduit, wherein the body is provided with an electromagnetic resonant cavity, operating at frequencies up to and including microwave frequencies, with resonant properties sensitive to the presence of a solid phase deposited on a conduit surface;
exciting an electromagnetic resonance of the cavity; and,
monitoring changes in the resonant properties of the cavity over time and directly detecting solids deposition on the conduit surface in response to changes in the resonant properties of the cavity.

In one embodiment, the method may comprise positioning a plurality of said bodies at spaced intervals along a length of the conduit. In particular, said bodies may be positioned along the length of conduit that may be at risk of solids deposition. Alternatively, said body may be positioned downstream of a Joule-Thomson valve.

In another aspect, the disclosure provides a method of detecting a composition and/or thickness of a solid deposit in a conduit, the method comprising the steps of:
positioning a first body to define at least a segment of a flow path for a fluid in or contiguous with the conduit, wherein the first body is provided with a first electromagnetic resonant cavity, operating at frequencies up to and including microwave frequencies, with resonant properties sensitive to the presence of a solid phase deposited on a conduit surface;
positioning a second body adjacent to the first body to define at least an adjacent segment of the flow path for the fluid in or contiguous with the conduit, wherein the second body is provided with a second electromagnetic resonant cavity, operating at frequencies up to and including microwave frequencies with resonant properties sensitive to the presence of the solid phase deposited on the conduit surface, wherein said resonant properties are different from the first electromagnetic resonant cavity;
exciting an electromagnetic resonance of the first and second cavities;
monitoring changes in the resonant properties of the first and second cavities over time and directly detecting solids deposition on the conduit surface in response to detected changes in the resonant properties of the first and second cavities;
assuming the solid deposit detected by the first and second bodies is the same composition and thickness, comparing the differences between the changes in resonant properties of the first and second cavities and determining the composition and/or thickness of the solid deposit based on the different response of the first and second cavities.

In one embodiment, the first electromagnetic resonant cavity has different dimensions to the second electromagnetic resonant cavity.

In an alternative aspect, the disclosure provides a method of detecting a composition and/or thickness of a solid deposit in a conduit, the method comprising the steps of:
positioning a body to define at least a segment of a flow path for a fluid in or contiguous with the conduit, wherein the body is provided with an electromagnetic resonant cavity, operating at frequencies up to and including microwave frequencies, with resonant properties sensitive to the presence of a solid phase deposited on a conduit surface;
exciting an electromagnetic resonance of the body in a first mode and subsequently in a second mode, wherein the first mode has a different electric field distribution within the conduit than the second mode;
monitoring changes in the resonant properties of the cavity when excited in the first and second modes and comparing the differences between the changes in resonant properties of the cavity when excited in the first and second modes to determine the composition and/or thickness of the solid deposit.

In a further aspect, the disclosure provides a method of detecting a dew point of a gas mixture in a conduit, the method comprising the steps of:
positioning a body to define at least a segment of a flow path for a fluid in or contiguous with the conduit, wherein the body is provided with an electromagnetic resonant cavity, operating at frequencies up to and including microwave frequencies, with resonant properties sensitive to the presence of a liquid phase condensed on a conduit surface;
exciting an electromagnetic resonance of the cavity; and,
monitoring changes in the resonant properties of the cavity over time and directly detecting liquid condensation on the conduit surface in response to changes in the resonant properties of the cavity.

### BRIEF DESCRIPTION OF DRAWINGS

Notwithstanding any other forms which may fall within the scope of the process as set forth in the Summary, specific embodiments will now be described with reference to the accompanying figures below:

FIG. 1 shows the pressure-temperature relationship for hydrate formation in natural gas at varying methanol compositions, representative of a subsea pipeline profile where approximately 70% of the pipe resides in a region of hydrate stability for an uninhibited system.

FIG. 2 is a schematic representation of a cross section of a pipeline undergoing hydrate film growth and subsequent plug formation.

FIGS. 3a and 3b are perspective cross-sectional and cross-sectional representations of one embodiment of an in-line sensor for detecting solids deposition in a conduit.

FIG. 4 is a cross-sectional representation of an alternative embodiment of the in-line sensor for detecting solids deposition in a conduit.

FIGS. 5a and 5b are perspective cross-sectional and cross-sectional representations of a further alternative embodiment of the in-line sensor for detecting solids deposition in a conduit.

FIG. 6 is a schematic representation of the respective electromagnetic cavities of a pair of adjacently positioned in-line sensors with respect to interrogation of a solids deposit in a conduit segment.

FIG. 7 shows respective graphical representations of relative dielectric permittivity measured by two in-line sensors as described herein positioned adjacent to one another with respect to interrogation of a solids deposit in a conduit segment.

FIG. 8 shows FEA simulated frequency shift for hydrate deposits of increasing size in methane at 0° C. and 40 bar. Two dimensions of growth were considered with increasing thickness into pipe (left) and increasing deposit length of fixed thickness (right). f_{gas only} is the frequency of the sensor containing methane only and f_{deposit} is the simulated frequency with the deposited layer.

FIG. 9 is a photograph of one embodiment of the sensor as described herein integrated into a 25.4 mm outer diameter conduit mounted on a wooden stand.

FIG. 10 is a schematic diagram of an experimental set up including the sensor as described herein and a fluid handling system.

FIG. 11 is a graphical representation of a hydrate equilibrium curve for water and carbon dioxide binary system calculated using CPA-Infochem models implemented in MultiFlash 6.2 with an experimental pathway shown by black arrow with 4 hold points from 10 to 4° C. inclusive.

FIG. 12 is a graphical representation of signal response of the sensor with time for a binary water carbon dioxide system operating at about 32 bar. Different experimental regimes are highlighted by colour transitions.

FIG. 13 is a graphical representation of experimental data showing the influence of hydrate formation on the signal response of the sensor. After detection, the system was isolated to observe hydrate deposit growth.

FIG. 14 is a graphical representation of the relationship between dielectric permittivity (εᵣ) and deposit thickness (δ) from a first sensor and an adjacent second sensor.

FIG. 15 is a graphical representation of the effect of changing split length and width of a sensor on the fractional electric field present in the flow path of the conduit as an indicator of electric field penetration.

FIG. 16 is a graphical representation of the relationship between dielectric permittivity (εᵣ) and deposit thickness (δ) from a sensor operated in a first mode and the sensor operated in a second mode.

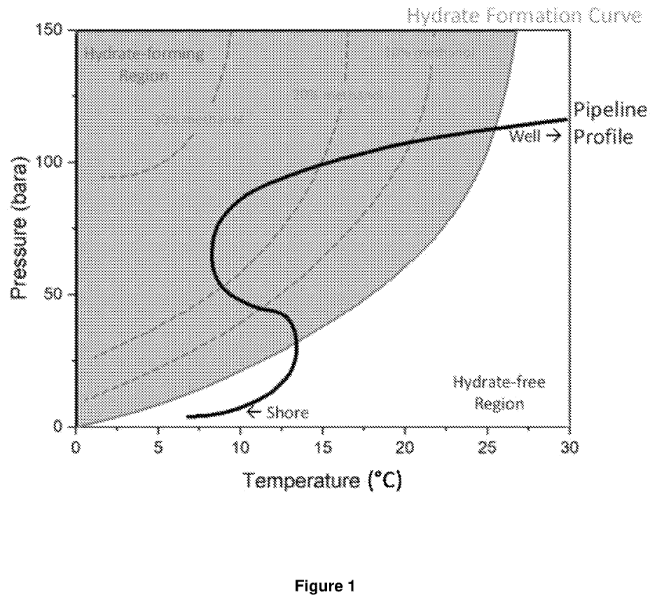

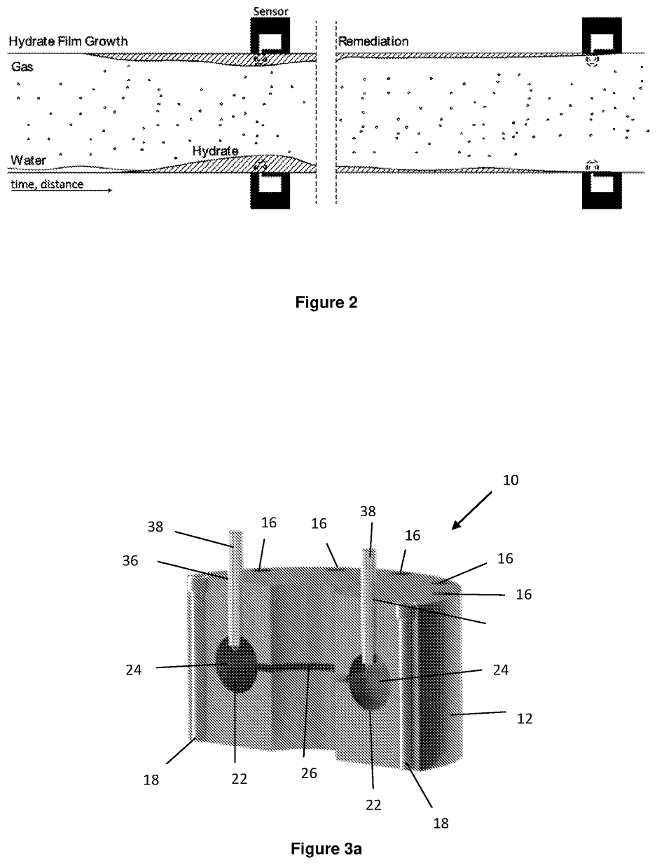

### DESCRIPTION OF EMBODIMENTS

The disclosure relates to an in-line solids sensor for detecting the solids deposits, such as methane hydrates, in pipelines.

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.).

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Reference to positional descriptions, such as lower and upper, are to be taken in context of the embodiments depicted in the figures, and are not to be taken as limiting the invention to the literal interpretation of the term but rather as would be understood by the skilled addressee.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “about” as used herein means within 5%, and more preferably within 1%, of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

### In-Line Solids Sensor

Embodiments described herein generally relate to an in-line solids sensor for detecting solids deposit, such as methane hydrates, in pipelines.

While the disclosure is made in the context of the formation of methane hydrates in natural gas pipelines, it will be appreciated that the disclosure has general application in the detection of deposits of wax or asphaltenes in pipelines, or even scale, fouling or biofouling. Other examples where the in-line solids sensor as described herein may have a general principle of application include, but are not limited to, detection of dew-points for carbon dioxide capture storage systems, detection of solid deposits or condensation in pipelines in food processing, water treatment, pharmaceuticals manufacture, chemical industries, pulp/paper manufacturing and so forth.

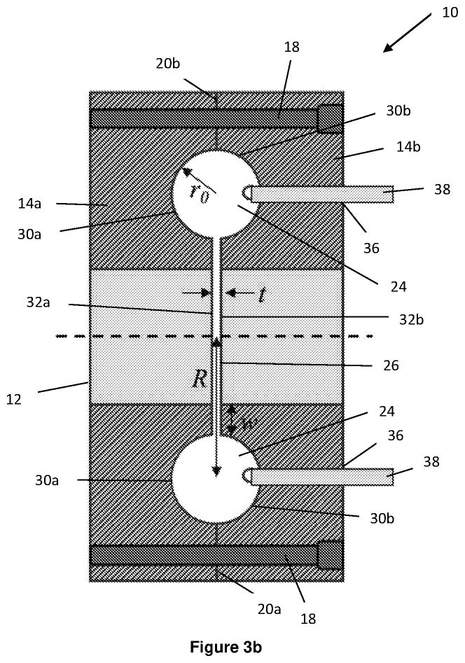

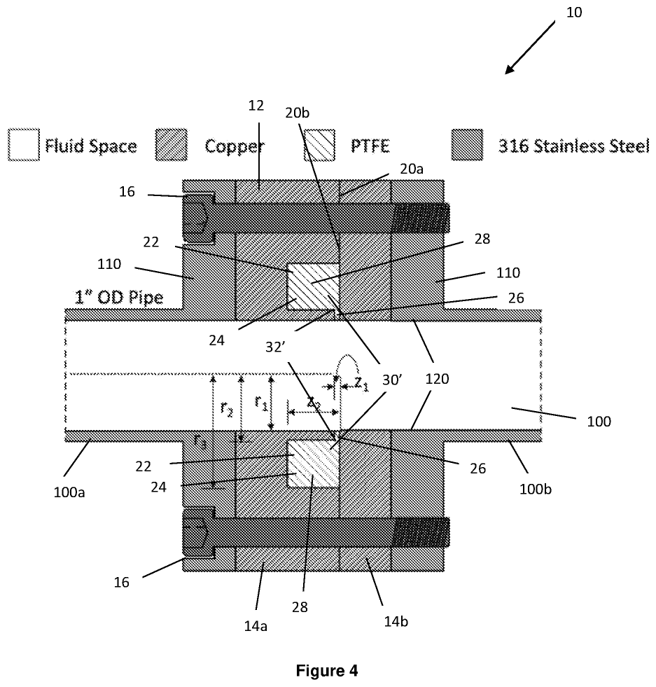

Referring to FIGS. 3a, 3b, 4, 5a, 5b, where like numerals refer to like features throughout, there are shown various embodiments of an in-line solids sensor 10 for directly detecting solids deposit in a conduit. Said sensor 10 is intended to be installed as a segment of a conduit 100 for fluid flow, for example a natural gas flowing through a subsea pipeline. Coupling arrangements at each end of said sensor 10 would vary according to the application but typically there may be respective flanges by which the sensor 10 might be clamped to complementary flanges 110 of further conduit segments 100a, 100b as shown in FIG. 4, or to the outlets or intake of pumping equipment, valves, elbows and so forth.

In the embodiments shown in FIGS. 3a, 3b and 4, the sensor 10 includes a body 12 positionable to define at least a segment of a flow path for a fluid in a conduit 100. The body 12 is formed by a pair of solid annular flanges 14a, 14b each contiguous with a respective cylindrical conduit section 100a, 100b. The flanges 14a, 14b have respective complementary through-holes 16 adjacent their outer peripheries for receiving respective bolts 18 and a sealing member, such as an O-ring, to clamp the flanges 14a, 14b together to form the assembled body 12. Opposing faces 20a, 20b of said flanges cooperate to define a cavity 22 when the flanges 14a, 14b are clamped together.

The body 12 may be fabricated from an electrically conductive material with a high thermal conductivity. Suitable examples include, but are not limited to, copper metal, aluminium, stainless steel.

In use, the cavity 22 behaves as an electromagnetic resonant cavity operating at frequencies up to and including microwave frequencies, with resonant properties sensitive to solids deposits as will be described later.

The cavity 22 includes annular chamber 24 having an annular groove 26 radially extending therefrom to an interior conduit surface 120. The width t of the annular groove 26 is typically less than the diameter or width of the annular chamber 24.

As shown in FIGS. 3a-3b, the annular chamber 24 may be toroidal having an internal radius ro. In this particular embodiment, each of the opposing faces of said flanges 14a, 14b are provided with a complementary outer annular circumferential recess 30a, 30b and an inner annular flat-sided recess 32a, 32b, so that when flanges 14a, 14b are assembled together, the annular chamber 24 and the annular groove 26 extending therefrom is defined in the body 12.

Alternatively, as shown in FIG. 4, the annular chamber 24 may have a square or rectangular cross-section. Although a square or rectangular cross-section may lead to a reduction in signal strength due to resistive losses in the sensor 10 in comparison to the toroidal annular chamber 24, the square or rectangular cross-section may be easier to fabricate with a suitable routing tool or cast. In this particular embodiment, the opposing face of one of said flanges 14b may be flat, and an outer annular recess 30′ and an inner annular recess 32′ may be provided in the opposing face of the other of said flanges 14a, so that when flanges 14a, 14b are assembled together, a square or rectangular chamber 24′ and the annular groove 26 extending therefrom is defined in the body 12. It will be appreciated that corners of the square or rectangular chamber 24′ may be rounded or curved by machining or a pressure seal to relieve some of the anticipated electrical losses associated with sharp junctions.

In other embodiments (not shown), the annular chamber 24 may be elliptical.

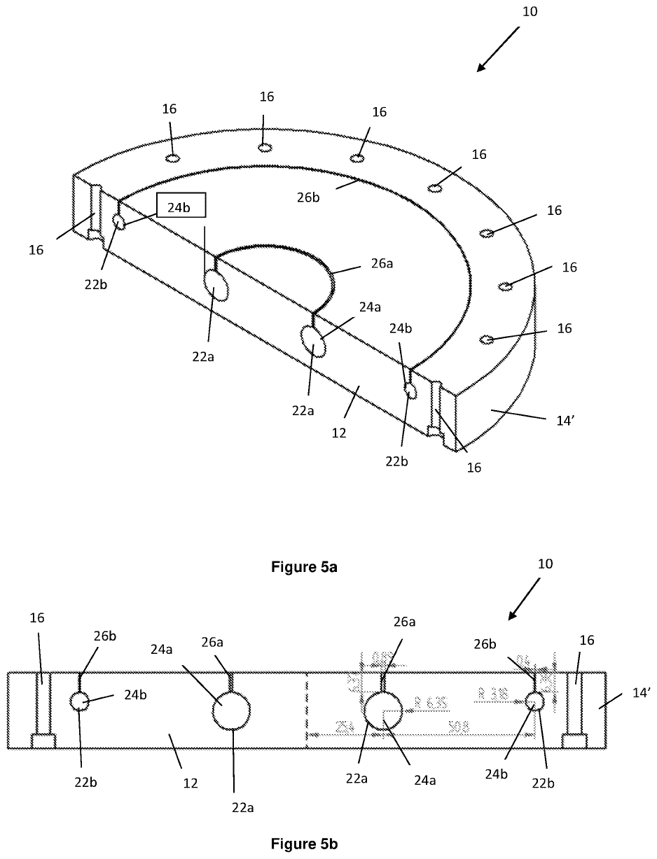

FIGS. 5a and 5b show a further embodiment wherein the body 12 includes a flange mountable plate 14′ having suitable through-holes 16 in said plate 14′ and matching bolts 18 to clamp the flange mountable plate 14′ to an end surface of the conduit 100.

In this particular embodiment, the flange mountable plate 14′ is provided with two concentric cavities 22a, 22b. Cavity 22a includes a first annular chamber 24a having a first annular groove 26a radially extending therefrom to an exposed plate surface 34 and a second concentric annular chamber 24b having a second annular groove 26b radially extending therefrom to the exposed plate surface 34. The second concentric annular chamber 24b is smaller than the first annular chamber 24a. The second annular groove 26b is the same depth as the first annular groove 26a but it is narrower than the first annular groove 26a. It will be appreciated that in other embodiments, there may be more than two concentric cavities.

It will be appreciated by those skilled in the art that the flanges 14a, 14b or the flange mountable plate 14′ may be fabricated by several conventional techniques including, but not limited to, casting, machining, stamping, forging and so forth.

In some alternative embodiments, the body 12 may comprise a toroidal body, wherein opposing sides of the toroidal body are contiguous with a respective conduit section. In this particular embodiment, the toroidal body 12 may be fabricated by a suitable 3D-printing technique whereby the cavity 22 in the body 12 is formed within the body 12 as it is being fabricated.

The cavity 22 in the body 12 may be filled with a sealing member 28 formed from a non-conductive material capable of forming a gas tight seal. The sealing member 28 may be marginally oversized and compressible to form the gas tight seal (i.e. >50 bar carbon dioxide). The sealing member 28 isolates the cavity 22 from external fluids and solids flowing through the conduit 100. Consequently, the body 12 of the sensor 10 offers no physical impedance to the pipeline geometry of the conduit surface 28 and prevents fluids from stagnating within the sensor 10. Suitable examples of non-conductive materials include, but are not limited to polytetrafluoroethylene (PTFE), polyether ether ketone (PEEK), neoprene, nitrile, viton, fluorosilicone, or glass-filled PTFE.

The body 12 is also provided with a plurality of ports 36 to receive one or more microwave probes 38 to excite and monitor an electromagnetic response of the cavity 22. The microwave probes 38 may include any suitable electromagnetic resonance sensor including, but not limited to, a frequency discriminator circuit (separate to or integral to an oscillator circuit), radiofrequency (RF) source and power sensor, or a network analyser. It will be appreciated that the microwave probes 38 may protrude into the sealing member 28 to provide sufficient coupling to a strong azimuthal magnetic field that is established in the annular chamber 24, as described below.

The sensor 10 may be in operative communication with a processor and a controller (not shown) which are used for data acquisition, resonant frequency measurement, and to generate real time data analysis of key parameters including, but not limited to, resonance frequency, temperature, pressure, and/or other associated sensors such as flow meters, moisture analysers, gas chromatographs or other sensors for compositional analysis, pH meter, and so forth.

As shown in FIG. 3a, when the microwave probes 38 are actuated to excite the cavity 22, a magnetic field B is established in the annular chamber 24 and an electrical field E is established in the annular groove 26. The electric field extends beyond the confines of the sealing member 28 and into the conduit 100 and is thus capable of investigating changing physical conditions in the immediate vicinity, for example solid deposits from 10 μm to 8 mm in height and width from the groove 26 at the conduit surface 120. It will be appreciated that some embodiments may be capable of detecting solid deposits which are less than 10 μm and greater than 8 mm in height and width from the groove 26 at the conduit surface 120. The magnitude of the electric field in the groove 26 (and the sensitivity of the sensor 10) may be changed by varying the width t of the groove 26.

The contents of the conduit 100 have negligible effect on the magnetic field B in the annular chamber 24 because the majority of materials and gas components in the conduit 100 have negligible permeability. Additionally, the contents of the conduit 100 have negligible effect on the magnetic field B in the annular chamber 24 because they are separated from, and thus do not interact with, the magnetic field B.

Various electromagnetic models, analytic and/or numerical in nature can be used to predict the resonant modes of the cavity 22. For example, in this particular embodiment, finite element analysis (FEA) may be used to solve electromagnetic field equations, allowing the frequency response to changes in dielectric permittivity (for example as associated with a fluid-phase transition) to be accurately modelled.

The groove 26 defines a capacitive region whereby the electrical field therein is concentrated, thereby making the resonant frequency of said cavity 22 highly sensitive to the relative dielectric permittivity (Er) of materials in the conduit 100, in particular overlying or proximal to the groove 26.

$${ε}_{r} = {( \frac{{f}_{0}}{f} )}^{2}$$

Consequently, the sensor 10 is arranged to monitor effective dielectric permittivity (ε_{eff}) of the contents of the conduit 100 over time rather than an absolute value of dielectric permittivity. Effective dielectric permittivity may be expressed as:

$${ε}_{eff} = {ε}_{eff}^{′} + {ε}_{eff}^{″} i$$

where ε_{eff} is a measure of the electric losses within a material and i=\sqrt{−1}. For the purposes of detecting changing conditions within the conduit 100, it is sufficient to monitor the real and imaginary components of the effective relative dielectric permittivity ε_{eff} using Equations. 1 and 2 respectively

$${ε}_{eff}^{′} = {( \frac{{f}^{0}}{f} )}^{2} \qquad ( 1 )$$

$${ε}_{eff}^{″} = \frac{{ε}_{eff}^{′}}{Q} \qquad ( 2 )$$

Here f⁰ and f are the resonant frequencies of the sensor 10 with the conduit 100 evacuated (sealing material still contained) and when interacting with a sample respectively. Q is the quality factor of the resonance frequency and is continually monitored.

The electric and magnetic field distributions in the sensor 10, associated with the resonant frequency f of the sensor 10, may be quantified and visualized using finite elemental analysis (FEA), such as by using Maxwell's equations implemented in COMSOL Multiphysics.

Generally, it is preferable if the dielectric permittivity of common components in the conduit 100 are distinctly different so that the magnitude and direction of change in relative dielectric permittivity will be indicative of a phase transition and phase type. For example, liquid water would increase the relative dielectric permittivity from about 1 (methane) to 80, whereas subsequent formation of a methane hydrate deposit would decrease the relative dielectric permittivity from 80 to 3.1.

Advantageously, the sensor 10 provides high sensitivity to the presence of a newly formed phase which has been shown to be a precursor to plugging mechanisms. Enhanced sensitivity to initial formation of a solids deposit would provide operators with an increased window of time in which to execute remediation measures (such as the introduction of chemical inhibitors or otherwise) before the solids deposit grows to an extent that would impede fluid flow or increase risk of a blockage. After remediation measures have been taken, the sensor 10 may also be used to quantify the effectiveness of the remediation measures, potentially avoiding the need for over-conservative dosing of chemical inhibitors.

In use, one or more sensors 10 may be positioned at spaced intervals along a length of the conduit 100, in particular along the length of conduit 100 that may be at risk of solids deposition.

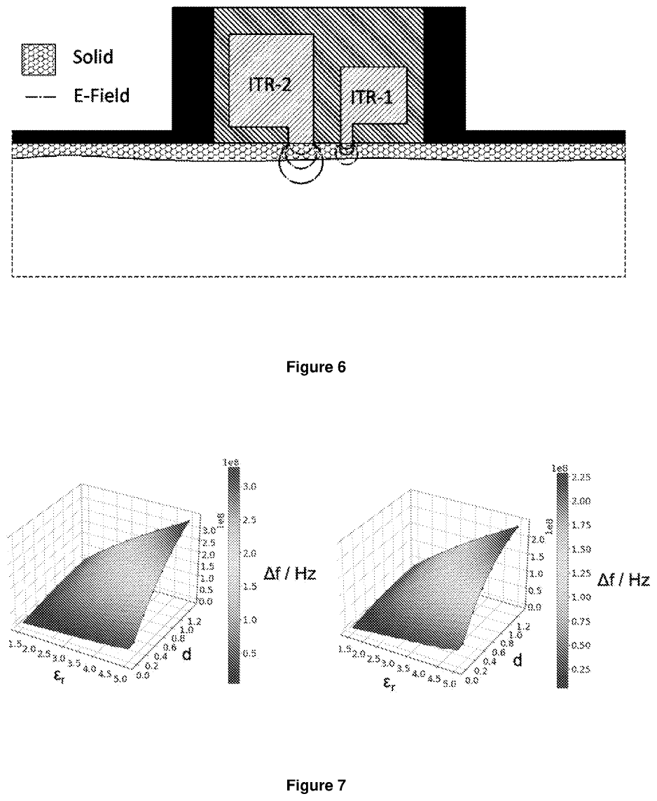

It may also be possible to determine a composition of a solid deposit in the conduit 100 using a pair of sensors 10. As shown in FIG. 6, a first body 12 may be positioned to define at least a segment of a flow path for a fluid in the conduit 100 and a second body 12′ may be similarly positioned to define an adjacent segment of the flow path for the fluid in the conduit 100. The second body 12′ has an electromagnetic resonant cavity that operates at different resonant properties than the electromagnetic resonant cavity of the first body 12′. Typically, this may be achieved by ensuring that the internal dimensions of one of the electromagnetic resonant cavities is smaller than the other. The microwave probes may then be actuated to excite the respective cavities. Changes in the resonant properties of the first and second cavities are monitored over time with solids deposition on the conduit surface 120 being directly detected by virtue of changes in the resonant properties of the first and second cavities. Using the assumption that the solid deposit that has been detected by the first and second resonant cavities has the same thickness and composition, the differences between the changes in resonant properties of the first and second cavities as shown in FIG. 7 may be used to determine the composition of the solid deposit.

Alternatively, the composition of the solid deposit in the conduit 100 may be interrogated using a single sensor 10 operating in two or more different modes. The different modes may be distinguished by different baseline frequencies. For example, the single sensor 10 may be excited at a baseline frequency of 2.035 GHz and then subsequently at a higher frequency of 10.6 GHZ, the effect being to establish a different electric field in the flow paths of the conduit 100. The changes in the resonant properties of the cavity when excited in the first and second modes may then be monitored and compared to determine the composition and thickness of the solid deposit.

Various embodiments may be illustrated by the following examples. The examples are provided for illustrative purposes only and are not to be construed as limiting the scope or content of the disclosure in any way.

### Example 1

A KEYSIGHT E5071C (range: 9 kHz to 4.5 GHZ) vector network analyser (VNA) with internal reference stability of +7 ppm was used to excite, monitor, and track the resonant frequency in real-time. The VNA measures the complex S₂₁ transmission coefficients across 401 stimulus frequencies spanning a range of 4 half-widths centred on the resonant peak. The maximum sweep time was 8 seconds, and the intermediate frequency bandwidth was 100 Hz; no averaging of the sweeps was performed. The resonance frequency and Q-factor were determined by regression of the complex S₂₁ data measured during the sweep as per conventional methods. Table 1 lists the measured and simulated vacuum frequencies and quality factors (Q-factors) for the in-line sensor at two conditions: vacuum, and 30 bar carbon dioxide at 10° C. The material surfaces were modelled with dimensions matching the fabricated sensor and conductivities of 1.35×10⁶ and 5.998×10⁷ S·m⁻¹ for the 316 stainless steel and copper respectively. Dielectric properties of the carbon dioxide (εᵣ=1.037) was calculated using the Harvey and Lemmon model implemented in REFPROP 10.0. for the measured temperature and pressure. The PTFE complex dielectric value is estimated to be εᵣ=2.06+0.000618i²⁵ however, there is apparent variability in this value depending on the plastic manufacturer.

TABLE 1
Measured and calculated (by finite-element analysis) resonant
frequencies and Q-factors for the fundamental sensor resonance
in a vacuum and 30 bara, 10° C. carbon dioxide.

| | | | Carbon Dioxide | |
|---|---|---|---|---|
| | Vacuum | | [30 bara, 10° C.] | |
| | Frequency/GHz | Q-Factor | Frequency/GHz | Q - Factor |
| Measured | 2.0428 | 701 | 2.0333 | 701 |
| Simulated | 2.0221 | 1760 | 2.0174 | 1763 |

Discrepancies in the simulated and measured frequencies can be attributed to minor geometric differences between the FEA model and physical cavity or differences in the assumed and actual PTFE dielectric properties. Tight tolerances in the split feature of this resonator (Z₁ 1 mm gap) mean that minor variations in spacing have tangible effects on the operating frequency of the sensor. A simulated 100 micrometre reduction in the gap size constitutes a ~30 MHz decrease in resonant frequency. This covers the observed discrepancy and is within the machining tolerance for the sensor. Deviations in Q-factors could also be attributed to an incorrect estimation of the PTFE dielectric losses. Furthermore, increased resistive losses from cavity surfaces and possible loading from the microwave probes and external circuit are also contributors.

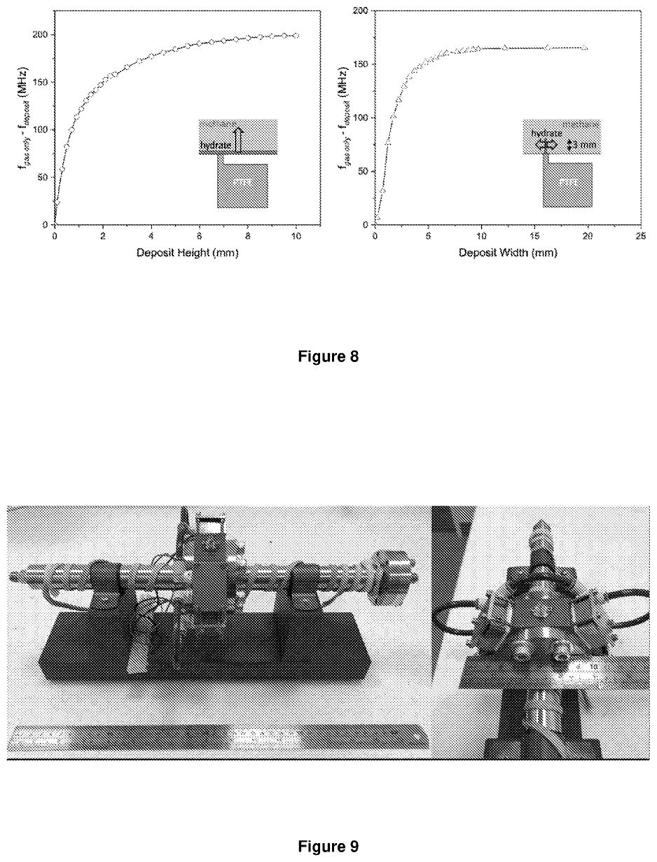

The sensors spatial sensitivity within the pipeline can be modelled using FEA by defining a pseudo-substance region that extends into the pipeline radially and lengthwise. For the example shown in FIG. 8 the system chosen was a hydrate deposit (εᵣ~3.15) developing in a 40 bar, 0° C. methane gas system. Two cases were considered: a deposit of fixed length with increasing thickness into the pipeline, and a deposit of fixed thickness increasing lengthwise along the pipe wall. As a conservative estimate we assume the minimum detectable shift to be one half-width of the resonant peak (~1.4 MHz) however measurement stability is typically on the order of 100 times better. The corresponding simulations suggests spatial sensitivity of approximately 8 mm in both directions with a minimum detection limit of ~100 microns. These predictions implicitly assume a uniform hydrate layer around the entire circumference of the pipeline over the sensing surface.

### Example 2

FIG. 9 depicts a fabricated sensor. The temperature in the cavity is controlled by altering power applied to associated Peltier thermoelectric elements whose heat is dissipated using fluid blocks with circulated 5° C. water. To further localise the cooling to the sensor, the ~0.2 m segments of pipe either side of the sensor are trace heated with a fixed 30 W. The terminating end of the pipeline has a removable flange which facilitates inspections inside the assembly when removed from the experimental set-up. This allows for investigation of conditions inside the pipeline including solids formed and structural integrity.

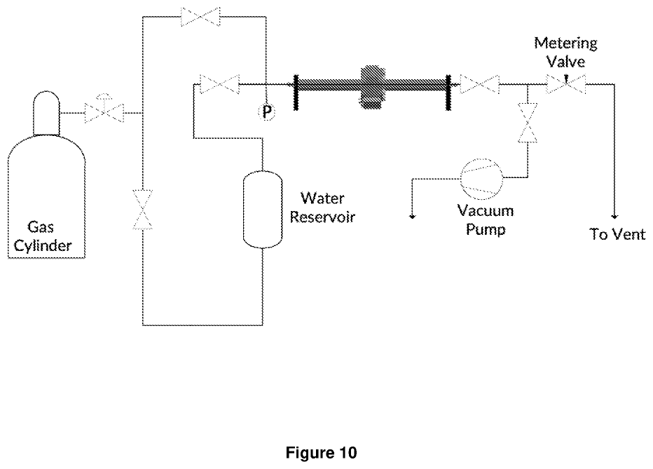

The sensor is integrated into a fluid handling system detailed in FIG. 10. During operation, carbon dioxide gas is flowed through one of two available paths prior to reaching the sensor. One path, here on in referred to as wet flow, passes through a water reservoir (Swagelok 316L-HDF4-300) half filled with de-ionized water allowing the gas to bubble through and partially saturate with water. The second path, dry flow, bypasses the water reservoir and directly flows through the sensor from the gas cylinder. The flow rate of each mode was controlled via a metering valve downstream of the sensor and was measured using a mass flow meter (Alicat MCP-50SLPM) to be approximately 23 standard litres per minute during dry flow. This flow rate was not measured continuously to avoid water build up within the flow meter, however, the positioning of the downstream valves remained constant throughout the experiment. Fluid flow is sustained by constant pressure operation of the gas cylinder with the attached regulator adjusted to maintain a normal operating pressure of approximately 32 bar. Pressure was measured with a transducer (OMEGA Engineering MMA3.5 KV) and along with the temperature and frequency measurement systems discussed earlier, it is continually monitored with LabVIEW data acquisition implemented on a desktop computer.

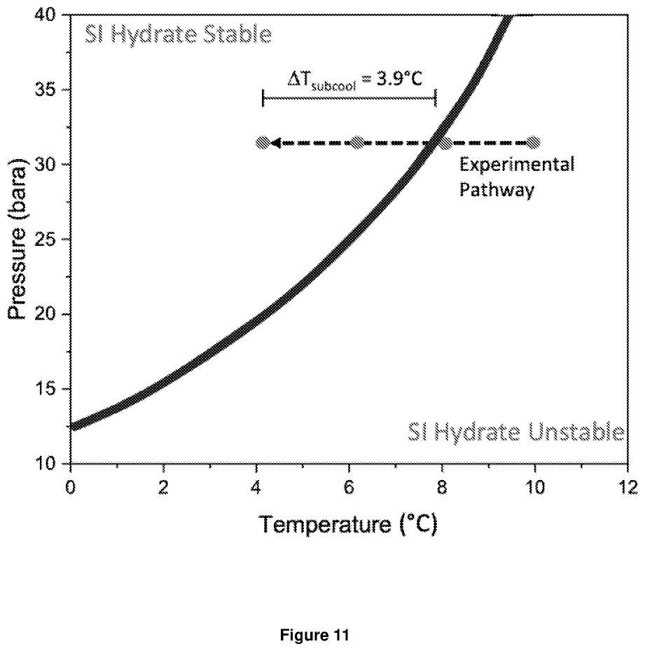

Four key operating regimes were used in the initial proof of concept experiment. The normal operating conditions were characterized via dry flow through the assembly with a constant 10° C. sensor temperature until a sufficient baseline frequency response had been established (50 minutes operation). Subsequent deviations from this normal operating frequency would thus be indicative of an upset condition. After 50 minutes, the flow mode was switched to wet flow where the room temperature, water saturated gas begins to condense on the sensing surface. Over 120 minutes, the temperature of the sensor was dropped in 2° C. increments (30-minute hold) from 10 to 4° C. following the experimental pathway into the hydrate equilibrium zone shown in FIG. 11. Once an initial hydrate deposit was detected, temperature was held constant at 4° C. for the remainder of the experiment, and the system was shut-in allowing for the remaining free water to convert to hydrate over approximately 1300 minutes. As the frequency signal converged on a steady signal from the hydrate deposit, the system was opened to dry flow to measure the remediating capacity of the dry carbon dioxide gas as it shifts the hydrate out of thermodynamic equilibrium temperature and sublimates the hydrate deposit. Due to limitations in available gas and the length of the experiment, the remediation could not be completed before losing pressure due to lack of material in the cylinder. It is envisioned that over sufficient time the signal would be restored to the baseline condition recorded in the first operating regime.

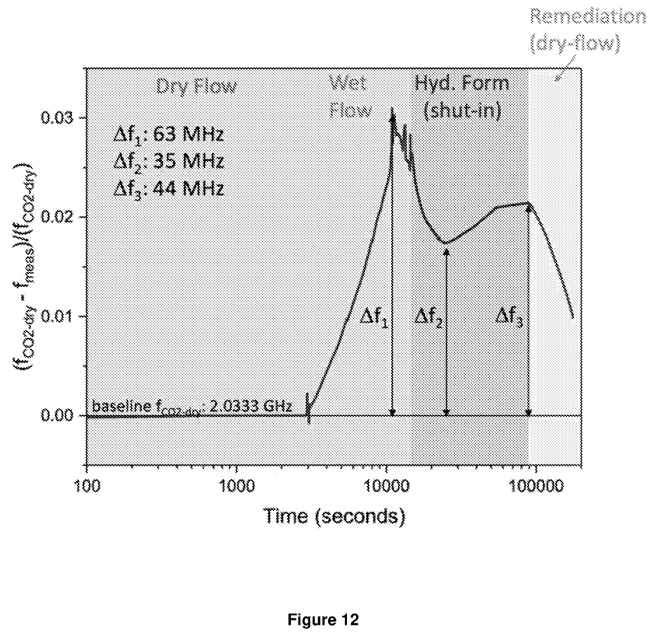

The signal response of the sensor was monitored throughout the entirety of the experiment and its relative deviation from the baseline frequency with time can be seen in FIG. 12. The vertical axis considers the average resonant frequency of the dry carbon dioxide flow (f_{CO2-dry}) and measured frequency (fₘₑₐₛ) only, however, they relate to dielectric permittivity via Eq. 3. Where ε_{eff-dry} is the average effective permittivity for the dry flowing system and ε_{eff} is the measured effective permittivity, both calculated using Equation 1 detailed above.

$$\frac{{f}_{CO 2 - dry} - {f}_{meas}}{{f}_{CO 2 - dry}} = \frac{{( {ε}_{eff - dry )} )}^{\frac{1}{2}} - {( {ε}_{eff} )}^{\frac{1}{2}}}{{( {ε}_{eff - dry )} )}^{\frac{1}{2}}} \qquad ( 3 )$$

On inspection, the transition from dry flow to wet flow corresponds to a gradual but appreciable downward shift in frequency of 63 MHz in the 120 minute time frame. The shift is correlated to the increasing appearance of dew on the sensor. The room temperature saturated gas passes the cold sensor, causing the water to condense on the internal surfaces of the copper, adjusting the effective permittivity of the volume investigated by the electric field. The direction and magnitude of the shift is expected due to the dielectric constant of water being significantly higher than the bulk carbon dioxide gas and thus increasing the effective permittivity.

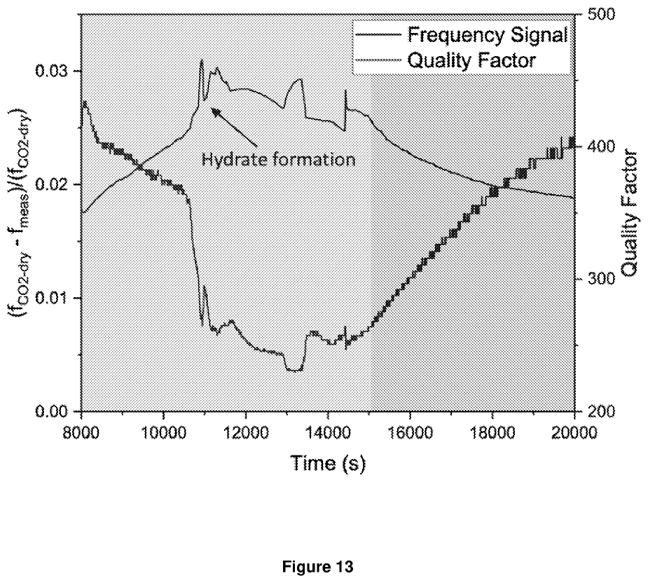

The onset of hydrate formation in this system is detected by a sharp change in signal response that is contrary to the gradual increase in effective permittivity observed in the wet flow operating regime. A subset of the data is shown in FIG. 13 below whereby the discontinuity in signal can be observed. Following the relationship established in Eq. 2, the quality factor is a direct indicator of dielectric losses within the system and can be seen to change with formation of the hydrate phase as a secondary confirmation of the new phase forming. The high dielectric loss aqueous material converts to hydrate, causing the signal quality factor to improve over time. During shut in, the frequency signal response partially recovered towards the baseline as the excess free water converted to hydrate. There is a local minimum in the signal highlighted by Δf₂ before an apparent increase in permittivity that begins to steady at Δf₃. It is suspected that between these two points, the hydrate deposit annealed, thus reducing the porosity of the deposit, and increasing the effective permittivity of the solid investigated by the sensor.

Upon introduction of the dry gas in the remediation regime, the system is shifted out of equilibrium by removing the water loaded carbon dioxide gas blanketing the hydrate. Using the CPA Infochem model implemented in Multiflash for a carbon dioxide+water system at operating conditions (~30 bar, 4° C.), the solid-vapour equilibrium (SVE) concentration of water in the gas phase is approximately 350 ppm. With the moisture specification of the dry carbon dioxide being less than 15 ppm (BOC laser grade), a thermodynamic driving force exists causing the hydrate to dissociate to recover the gas phase water concentration to equilibrium conditions. In this instance, the hydrate deposit does not dissociate into a free water phase, rather, it sublimates. This is confirmed by the sensor response as there is a continual recovery of signal towards the baseline with continued flow. If a free water phase were formed, a frequency response in the other direction would be expected due to the distinct contrast in dielectric properties of hydrate and water.

### Example 3

If the composition of the solid deposit is known (e.g., hydrates, waxes, etc.) then the thickness of the deposit can be estimated using FEA. For systems where the composition, and hence dielectric properties, of the solid deposit are unknown, it becomes increasingly difficult to distinguish between a small deposit of high dielectric material and a large deposit of material with a low dielectric constant. In this instance, the sensor 10 can exhibit the same signal behavior unless there are considerable differences in dielectric losses (e.g., water). While it is expected that experienced operators will have a good understanding of high-risk solid deposit composition, there are two methods in which the sensor 10 may be used to measure both deposit properties and thickness. Proof of concept of this technique is shown via FEA simulation studies.

The first method involves positioning a pair of sensors 10 in close proximity to each other with respect to a conduit surface 120. The first sensor has different dimensions to the second sensor. In particular, the width t of the annular groove of the first sensor may be smaller or larger than the width t of the annular groove of the second sensor. The dimensional differences between the annular chamber and the annular groove alter the resonance frequency of the cavity, in particular the dimensional differences of the width t of the annular grooves change the penetration of the electric field E into the fluid space of the conduit 100. If a larger percentage of the fringing electric field is interacting with the solid, as may be the case for the second sensor, then the relative frequency change will be larger than measured by the first sensor.

Assuming the solid deposit is uniform in thickness and in composition across the first and second sensors, there will be an optimal solution for the dielectric constant,

$${ε}_{r}^{*} ,$$

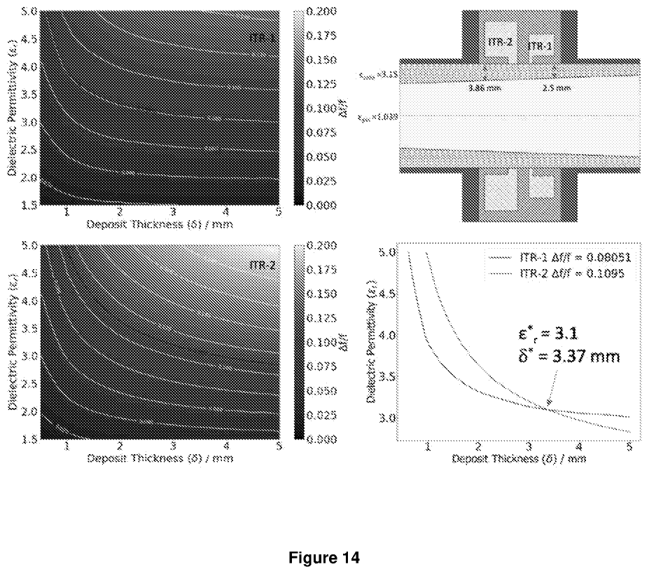

and thickness δ*, which would cause the observed frequency changes in the first and second sensors. Using FEA, it is possible to perform a parametric sweep across a range of deposit thickness and dielectric properties to generate a surface that is unique to each sensor. Ideally, the surface should be generated using experimental calibration however for the wide range of possible geometries, pipe diameters, and dielectric constants, it is recommended that FEA be used. FIG. 14 shows examples surface plots for two different sensor geometries where deposit thickness is modelled between 0.5 to 5 mm and dielectric constant between 1.5 and 5.0. The objective function detailed in Eq. 4 below where

$${ε}_{r}^{*}$$

and δ* are iterated along the FEA generated surface to find the optimal solution. This method captures the dielectric dependence that can sometimes act to perturb the electric field distribution.

$$O b j = {{Σ}_{j = 1}^{2} ( \frac{Δ {f}_{j , FEA}}{{f}_{j , FEA}} ( {ε}_{r}^{*} , {δ}^{*} ) - \frac{Δ {f}_{j , meas}}{{f}_{j , meas}} )}^{2} \qquad ( 4 )$$

The width of the annular groove is critical to field penetration, which may be increased by halving the width of the annular groove in comparison to the other sensor. This can alter the operating frequency of the sensor, however, the dimensions of the rectangular toroid section can be adjusted so frequencies match without significant influence on the fringing field. For the presented example, the baseline operating frequencies in the presence of 40 bar methane at 0° C. are 2.035 and 1.921 GHz for the first and second sensors respectively. FIG. 14 shows the simulated results of a case study where a sloping deposit of hydrate (εᵣ=3.15) is interacting with both sensors. For each sensor, there is a contour (black line) of many solutions for

$${ε}_{r}^{*}$$

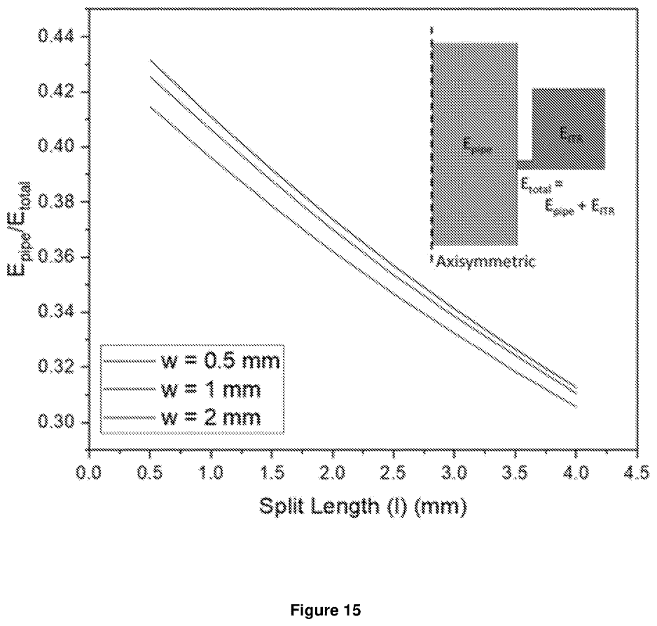

and δ* that can cause the observed Δf/f values. While individually there is no unique solution, overlaying them produces a single intersection and solution for the solid dielectric and thickness. Here the predicted dielectric differs by 0.05 and the thickness is an approximate average of the slope over the region of investigation. Implicit in this study, is the assumption that the solid deposit is uniform across the entire circumference of the pipeline. It will be appreciated that the pairing of the first and second sensors would be optimised based on the pipeline diameter and desired sensitivity to deposit thickness. Here, FEA presents as a powerful tool where design decisions can be efficiently tested as seen in FIG. 15 that explores the effect of changing split length and width a single sensor on the fractional electric field present in the fluid space as an indicator of field penetration. The electric field may be calculated by computing the volume integral (V·m²) of the field magnitude about the axis of symmetry.

### Example 4

Alternatively, the method for determining the composition and thickness of the solids deposit may involve operating a single sensor in different modes whereby the electric field distribution is altered at different resonant frequencies.

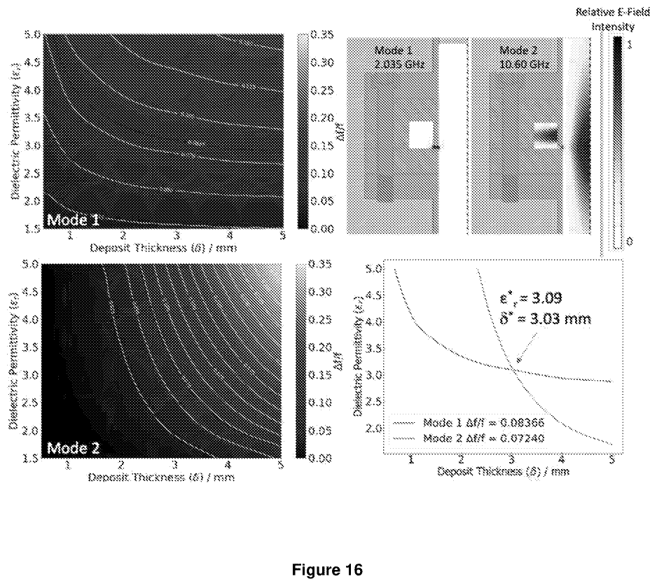

Referring to FIG. 16, the single sensor may be operated in a first mode at a baseline frequency of 2.035 GHz and in a second mode at a higher frequency of 10.60 GHz. The distribution of the electric field is different with 38.4% and 72.8% of the electric field existing in the fluid space for the first and second modes, respectively. In this example, a 3 mm thick hydrate layer was simulated, and the frequency response of each mode was calculated. Following the objective function, the intersection of the two Δf/f contours at

$${ε}_{r}^{*} = 3.09$$

and δ*=3.03 mm were in good agreement with the simulation inputs. Discrepancies were largely attributed to the interpolation of the surface as it was originally generated with coarse 0.5 increments in the dielectric constant and thickness.

To provide a robust solution to the objective function, it is important that there is sufficient contrast in sensor (or mode) responses to a solid deposit. The method which operates a single sensor in dual modes exhibits this large contrast but is limited by the second mode in regions where the Δf/f approaches 0. In this region (approx. deposit thickness 0 to 1.5 mm), the sensor only provides one, non-zero value of Δf/f for the first mode giving rise to a single contour of possible solutions for dielectric and thickness. This is an artefact of the chosen sensor dimensions and can be adjusted with geometry optimization to be sensitive to smaller deposits. For a given industrial application, it is envisioned that the geometry will be adjusted to probe a certain solid (dielectric) and thicknesses of operational interest. For improved versatility, the single sensor and the paired sensors can be used together. This application will yield several individual and unique responses to a solid deposit and can identify a distribution of solid thickness along a pipeline.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

In the claims which follow and in the preceding description except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. An in-line sensor for detecting solids deposition in a conduit, the sensor comprising:
a body positionable to define at least a segment of a flow path for a fluid in or contiguous with the conduit;
a cavity in the body defining an electromagnetic resonant cavity, operating at frequencies up to and including microwave frequencies, with resonant properties sensitive to the presence of a solid phase deposited on a conduit surface;
one or more probes for exciting and monitoring an electromagnetic resonance of the cavity; and
a signal processor operatively connected to said one or more probes to directly detect solids deposition on the conduit surface in response to detected changes in the resonant properties of the cavity.

2. The in-line sensor according to claim 1, wherein the cavity comprises an annular chamber having an annular groove radially extending therefrom to the conduit surface.

3. The in-line sensor according to claim 2, wherein the annular chamber is toroidal having an internal radius r₀.

4. The in-line sensor according to claim 2, wherein the annular chamber has a square or rectangular cross-section.

5. The in-line sensor according to claim 2, wherein a width t of the annular groove is shorter than the radius or the diameter of the annular chamber.

6. The in-line sensor according to claim 1, wherein said body comprises a pair of annular flanges each contiguous with a respective conduit section, wherein opposing faces of said flanges cooperate to define the cavity when the flanges are assembled together.

7. The in-line sensor according to claim 6, wherein the body comprises through-holes in the annular flanges and matching bolts are provided for clamping the flanges together.

8. The in-line sensor according to claim 6, wherein the opposing face of one of said flanges may be flat, an outer annular recess and an inner annular recess being provided in the opposing face of the other of said flanges.

9. The in-line sensor according to claim 6, wherein the opposing faces of said flanges are provided with an outer annular circumferential recess and an inner annular flat-sided recess.

10. The in-line sensor according to claim 1, wherein said body comprises a toroidal body, opposing sides of the toroidal body being contiguous with a respective conduit section.

11. The in-line sensor according to claim 1, wherein said body comprises a flange mountable plate having through-holes therein and matching bolts to clamp the flange mountable plate to an end surface of the conduit.

12. The in-line sensor according to claim 11, wherein said plate is provided with a plurality of concentric toroidal cavities.

13. The in-line sensor according to claim 12, wherein a first toroidal cavity comprises a first annular chamber having a first annular groove radially extending therefrom to an exposed plate surface and a second toroidal cavity comprises a second concentric annular chamber having a second annular groove radially extending therefrom to the exposed plate surface.

14. The in-line sensor according to claim 13, wherein the second concentric annular chamber has the same or different dimensions to the first annular chamber.

15. The in-line sensor according to claim 13, wherein the second annular groove has the same or different depth to the first annular groove.

16. The in-line sensor according to claim 13, wherein the second annular groove has the same or different width to the first annular groove.

17. The in-line sensor according to claim 1, wherein the cavity in the body is filled with a non-conductive material capable of forming a gas tight seal.

18. The in-line sensor according to claim 17, wherein the non-conductive material comprises PTFE, PEEK, neoprene, nitrile, viton, fluorosilicone or glass-filled PTFE.

19. The in-line sensor according to claim 1, wherein said sensor is capable of detecting a solids deposit having a height in a range of 10 μm to 8 mm and a width in a range of 10 μm to 8 mm.

20. The in-line sensor according to claim 1, wherein said signal processor is arranged to directly detect solids deposition on the conduit surface in response to detected changes to dielectric permittivity (Δε) with time.

21. (canceled)

22. (canceled)

23. (canceled)

24. (canceled)

25. (canceled)

26. (canceled)

27. (canceled)

28. (canceled)
